(19) United States
(12) Patent Application Publication
(10) Pub. No.: US 2026/0020847 A1
(43) Pub. Date: Jan. 22, 2026

(54) Vascular Closure Device for Shallow Vessels

(71) Applicant: Cyndrx, LLC, Brentwood, TN (US)

(72) Inventor: John I. Shipp, Jacksonville, FL (US)

(21) Appl. No.: 19/270,204

(22) Filed: Jul. 15, 2025

Related U.S. Application Data
(60) Provisional application No. 63/672,618, filed on Jul. 17, 2024.

(51) Int. Cl. A61B 17/00

(52) U.S. Cl. CPC A61B 17/0057; A61B 2017/00004; A61B 2017/00601; A61B 2017/00619; A61B 2017/00623; A61B 2017/00862

(57) ABSTRACT

A vessel seal assembly for sealing an opening in a wall of a blood vessel of an appendage such as the ulnar or radial artery, including a first sealing element for placing inside the lumen of the blood vessel, a shaft, the shaft fixed in a predetermined configuration relative to the first sealing element, a flexible member surrounding at least a portion of the shaft adjacent the first sealing element, and a second sealing element, the second sealing element slidingly movable relative to the first sealing element along the shaft to engage the exterior wall surface and configured to position the flexible member against the interior wall surface of the blood vessel to seal the opening in the blood vessel, the shaft and the first sealing element are joined at an angle between about 20 and 30 degrees.

### REFERENCE TO RELATED CASES

This application claims priority under 35 U.S.C. § 119(e) to U.S. Provisional Patent Application Ser. No. 63/672,618 filed Jul. 17, 2024, the contents of all of which are incorporated by reference.

### BACKGROUND OF THE INVENTION

Human arteries and veins are accessed for a variety of diagnostic and interventional procedures. Vascular access is gained by the well-known Seldinger or modified Seldinger technique using a needle, guidewire, and an access sheath, comprising a hub and cannula. The insertion angle of the access needle depends on the depth of the lumen. The femoral artery depth, for example, varies from 10-60 mm below skin level depending on body mass index (BMI) of the patient. The needle angle for femoral artery access varies between about 25 and 75 degrees, depending on BMI, higher angles for larger BMIs.

Radial or ulnar artery (appendage) access is sometimes indicated. The depth of the radial artery varies from 2-3 mm and a much lower needle access angle is therefore employed, usually in the range of 15-30 degrees.

After completion of a procedure and removal of the access sheath, it is necessary to close the access wound. The gold standard for vessel closure is manual compression (“MC”), which requires holding considerable pressure upstream of the lumen access site so that bleeding is finally arrested owing to the clotting cascade. MC has many shortcomings. It is time consuming (up to 30 minutes), it usually requires reversal of antithrombin medication employed during the procedure, it can result in rebleeding after initial hemostasis, and it can result in nerve damage, usually treated with opioids.

To overcome some of the MC issues vascular closure devices (“VCDs”) were designed and disclosed in U.S. Pat. Nos. 6,045,569; 8,945,178; and 11,253,242, for example. The modality of these VCDs are coagulants, suture mediated devices or mechanical compression devices. The coagulant elements comprise extravascular components of either bovine collagen or polyethylene glycol (PEG) with or without intra-vascular elements. Removal of the sheath allows blood to exit the wound into the sheath tissue track where it contacts the coagulant material. The coagulant swells from the absorbed blood thus forming a barrier that over time provides hemostasis. Since the wound is required to bleed before exhibiting hemostasis, these devices are merely hemostasis assist devices rather than true seals. As a result, this category of VCDs results in a high incidence of hematoma formation. To counter the hematoma events, antithrombin medications are usually required. The coagulants solicit an inflammatory foreign body response that leaves scarring that can obviate re-cannulation that may be required. Malfunction or incorrect deployment of these devices can expose the blood stream to the highly thrombogenic coagulant. The time to hemostasis (“TTH”) of these devices is better than MC, in the 2 to 6-minutes time frame.

There are two subclasses of mechanical compression devices (“MCDs”). One uses a tourniquet that operates in the same modality of MC in that large forces are applied over a large area resulting in a counter-pressure to the local blood pressure. Examples are disclosed in U.S. Pat. Nos. 8,524,974 and 6,264,673. These devices are a direct substitute for MC applied by the hands of a doctor or trained technician. As such, they suffer from the same shortcomings, long “TTH” and frequent nerve damage. The tourniquet area of the '947 radial device is approximately 1,000 mm² usually encompassing a portion of the radial nerve causing pain and sometimes nerve damage.

The second MCD category is disclosed in U.S. Pat. No. 11,253,242 ('242 patent), the contents of which are incorporated by reference. The device in the '242 patent comprises an extravascular component called a floating foot (“FF”) and an intravascular distal foot (or footplate) with an integral shaft that extends through the wound. The integral shaft minimizes the likelihood of embolization of the intravascular element. The shaft comprises a distal end integral with the distal foot and a proximal end separated by a notch feature that is configured to break the seal away from the seal applier when a sufficient sandwich force is reached.

A latching component called a proximal seal (“PS”) abuts the FF and complimentary latching elements on the shaft and PS robustly sandwiches intra and outer seal components together thus providing instant mechanical compression hemostasis with the force distributed over a small area, thereby avoiding nerve contact and pain.

The distal foot width is slightly less than the cannula ID and its length is usually 1.75-3× of the cannula OD to provide ample footplate purchase against the interior vessel wall. In the deployed state the distal foot opposes and is parallel to the FF. Inside the cannula the FF is at an angle of about 25 degrees relative to the longitudinal axis of the cannula and rotates to 45 degrees and becomes parallel to the foot as the PS advances distally and the sandwich is sealed.

A PS or latch creates the sandwich with the DS and FF on either side of the vessel wall thus provides an instant mechanical seal. In another embodiment, a flexible gasket is configured between the DS and the inside vessel wall to provide sealing over a large wound in case of wounds that may exceed expected proportions, particularly for mid and large bore access. Since this device is primarily indicated for femoral artery or vein access the vessel depth dictates that the access needle entry angle averages 45 degrees the integral shaft is formed at 45 degrees relative to the vessel contact surface of the DS.

For the small-bore devices disclosed in the '242 patent, the cannula of the sheath has an inside diameter (“ID”) of approximately 2 mm (6 French), for example, and an outside diameter (“OD”) of 2.7 mm. Since the foot-to-shaft angle is 45 degrees the distal foot with the integral shaft element must be flexible and elastic enough to deform during the insertion process, owing to the necessary length of the distal foot, such that the DS returns to its equilibrium shape upon entering the blood stream, as disclosed in the '242 patent. The need for the vessel seal assembly to remain within the elastic limit of the absorbable polymer throughout the insertion process puts severe restraints on the vessel seal assembly design.

What is needed then for shallow vessel access such as the radial or ulnar artery is an integral foot and shaft configured such that upon insertion into the access cannula the integral foot and shaft readily pass through the cannula with little friction and lack of deformation in conjunction with an extra vascular element to achieve instant hemostasis without nerve damage. What is also needed is a method of sealing a shallow vessel resulting in instant hemostasis without nerve damage.

### SUMMARY OF THE INVENTION

The present invention is directed to a vessel seal assembly for sealing an opening in a wall of a blood vessel of an appendage such as the ulnar or radial artery, the blood vessel having an interior wall surface, exterior wall surface, and a lumen, the vessel seal assembly includes a first sealing element for placing inside the lumen of the blood vessel, a shaft formed with the first sealing element as a single one-piece component, the shaft fixed in a predetermined configuration relative to the first sealing element, the shaft having a length sufficient to extend through the opening of the blood vessel and at least a portion of any tissue overlying the blood vessel, a flexible member surrounding at least a portion of the shaft adjacent the first sealing element, and a second sealing element, the second sealing element slidingly movable relative to the first sealing element along the shaft to engage the exterior wall surface and configured to position the flexible member against the interior wall surface of the blood vessel to seal the opening in the blood vessel.

In some embodiments, the shaft and the first sealing element are joined at an angle between about 20 and 30 degrees.

In some embodiments, the shaft and the first sealing element are joined at an angle of about 25 degrees.

In some embodiments, the second sealing element is configured to position the flexible member and the first sealing element against the interior wall surface of the blood vessel to seal the opening in the blood vessel.

In some embodiments, the flexible member is connected to the first sealing element.

In some embodiments, the flexible member is secured to a proximally facing surface of the first sealing element.

In some embodiments, the flexible member is substantially circular and has an opening in a middle portion thereof to receive the shaft therethrough.

In some embodiments, the flexible member has a shape selected from the shapes consisting of round and oval.

In some embodiments, the shaft has a plurality of equally spaced projections along a length thereof.

In some embodiments, the shaft has a reduced portion, the reduced portion having a cross section being smaller than a cross section of any other portion of the shaft.

In some embodiments, the shaft has a plurality of equally spaced projections along a length thereof and the shaft has a reduced portion, the reduced portion having a cross section being smaller than a cross section of any other portion of the shaft.

In some embodiments, the first sealing element, the shaft, the outer floating element, and the second sealing element are made from a bio-absorbable material.

In some embodiments, the shaft breaks at the reduced portion as a result of a force exerted on the second sealing element, pushing the second sealing element against the blood vessel and the flexible member and the first sealing element.

In some embodiments, the vertical distance from the reduced portion to a distal surface on the first sealing element is less than 5 mm.

In some embodiments, the reduced portion is disposed between two of the plurality of equally spaced projections.

In some embodiments, the second sealing element is latched into place when the reduced portion breaks, thus forming a sandwich force between the first sealing element and the second sealing element.

In some embodiments, the sandwich force is dictated by strength of material and dimensions of the notch and the sandwich force between the first sealing element and the second sealing element is independent of a user or anatomy.

A vessel seal assembly for sealing a vessel wound (lumenotomy) that is near the skin level such as a radial or ulnar artery is presented. The vessel has an exterior wall surface, an interior wall surface, and a lumen, the vessel seal assembly comprises a first sealing element with a distal and a proximal surface placing inside the lumen integral with a shaft extending from the proximal surface of the first sealing element at an acute angle, preferably about 25 degrees, and extending through the lumenotomy and into a tissue track formed by the insertion of an access cannula by the well-known Seldinger or modified Seldinger technique.

The vessel seal assembly comprises two states, the home state whereby a second sealing element, having a distal surface and a proximal surface and a passage configured to allow the second seal element to be slidable attached to the shaft at a predetermined distance proximal to the lumenotomy. The distal surface of the second sealing element is preferably about 25 degrees with respect to the shaft's longitudinal axis such that in the second state or seal state the distal surface of the second seal element is in contact with the outer wall of the lumen and is parallel with the proximal surface of the first sealing element. The distal surface of the second sealing element remains at about 25 degrees angle with respect to the longitudinal axis in either state and does not rotate or vary when moved from the home state to the seal state. The parallel configuration between the first and second sealing elements is constant in the home state and the seal state, therefore no FF element is needed as disclosed in the '242 patent.

The shaft and the passage of the second sealing component are configured with latching elements that maintain a sandwich force between the first and second sealing elements which results in instant hemostasis.

An elastic, flexible gasket, having a distal and a proximal surface is configured such that a portion of the distal surface of the gasket contacts the proximal surface of the first sealing element and proximal surface of the gasket contacts the interior wall of the vessel to assure ample seal coverage about the lumenotomy. The distal and proximal surface area of the gasket increases with increased closure French size. For example, the area is preferably 9 mm² and 50 mm² for 6 and 14 French, respectively. These small areas and the weakness of the sandwich force minimize the possibility of nerve damage, while providing adequate pressure for hemostasis and robustness without tissue necrosis.

Also, disclosed herein is a method of sealing an arteriotomy of a shallow vessel such as radial or ulnar artery providing instant hemostasis without causing nerve damage. Access is gained by well-known methods for a vascular procedure, either diagnostic or interventional. After the procedure the VCD of this invention is inserted through the sheath which is inserted into the wrist area at an angle, preferably 25 degrees, into the target artery via the sheath. The sheath and VCD are retracted to the point whereby the gasket contacts the interior wall of the artery at the arteriotomy. The PS is moved distally past the break notch, until it abuts the exterior of the vessel wall at the arteriotomy with enough force to cause the break notch (BN) to break, thereby providing instant hemostasis. Latch features on the shaft and PS hold the sandwich force, (equal to the force required to break the BN), in place until the seal integrity gives way to absorption, long after the arteriotomy has healed and the temporary seal is no longer needed.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a perspective view of one embodiment of a vessel seal assembly according to the present invention in an exploded state;

FIG. 2 is a side elevational view of the vessel seal assembly in FIG. 1;

FIG. 3 is a top view of a shaft used with the vessel seal assembly in FIG. 1 in partial cross section;

FIG. 4 is a cross section of a second sealing element along the line 4A-4A in FIG. 5;

FIG. 5 is a cross section of the second sealing element orthogonal to the shaft;

FIG. 6 is a cross section of the second sealing element along its length;

FIG. 7 is a cross section of the vessel seal assembly in a sheath with a holding tube and pusher;

FIG. 8 is a perspective view of the vessel seal assembly of FIG. 1 inserted in a vessel of the body in the home state;

FIG. 9 is perspective view of the vessel seal assembly of FIG. 1 inserted in a vessel of the body with distal foot against the interior vessel wall; and

FIG. 10 is a perspective view of the vessel seal assembly of FIG. 1 in a sealing state.

### DETAILED DESCRIPTION OF THE PREFERRED EMBODIMENTS

Reference will now be made in detail to the present preferred embodiment(s) of the invention, examples of which are illustrated in the accompanying drawings. Whenever possible, the same reference numerals will be used throughout the drawings to refer to the same or like parts.

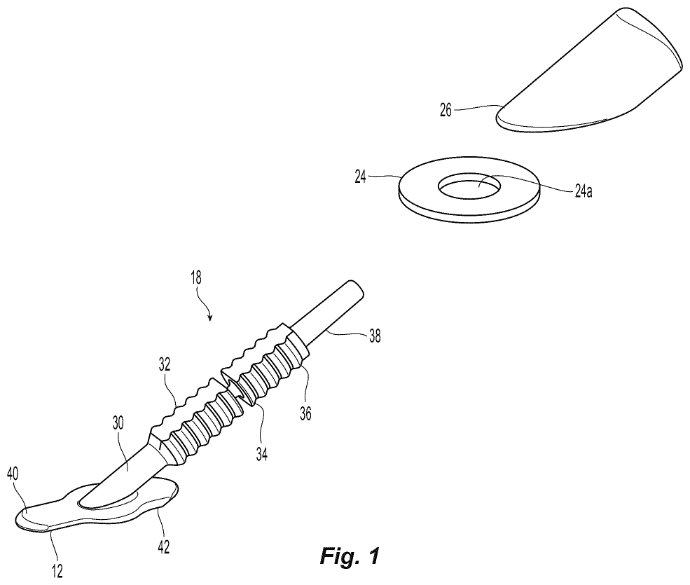

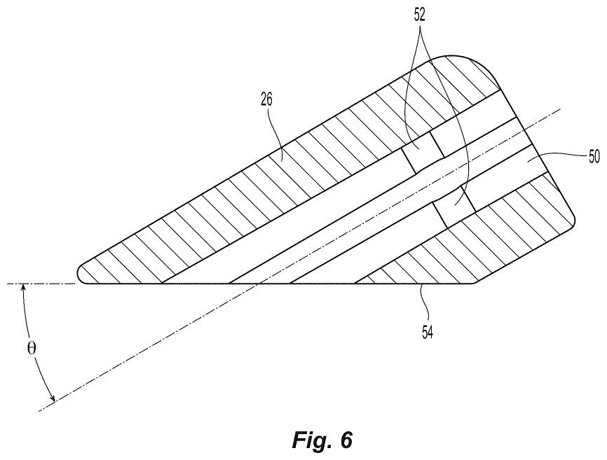

Turning now to FIGS. 1-6 is an exploded view of one embodiment of the vessel seal assembly 10 for sealing an opening in a wall of a blood vessel of an appendage such as the ulnar or radial artery. The vessel seal assembly 10 could also be used in a leg for the femoral artery. The vessel seal assembly 10 preferably includes a first sealing element or foot 12 for placing inside the lumen 14 of the blood vessel 16. The blood vessel 16 has an interior wall surface 16a and an exterior wall surface 16b. The vessel seal assembly 10 also includes a rigid shaft 18 formed with the first sealing element 12 as a single one-piece component, the shaft 18 fixed in a predetermined configuration relative to the first sealing element 12, the shaft 18 having a length sufficient to extend through the opening 20 of the blood vessel 16 and at least a portion of any tissue overlying the blood vessel 16. The vessel seal assembly 10 also has a flexible member or gasket 24 surrounding at least a portion of the shaft 18 adjacent the first sealing element 12. The flexible member 24 may also be connected to the first sealing element 12 by securing it to a proximally facing surface of the first sealing element 12. In one embodiment, the flexible member 24 is substantially circular (it may also be oval or have another configuration) and has an opening 24a in a middle portion thereof to receive the shaft 18 therethrough.

The vessel seal assembly 10 also has a second sealing element or proximal seal 26.

Shaft 18 includes an ankle 30 that joins the foot 12 to the distal latches 32, as well as a break notch 34, proximal latches 36, and holding taper 38. It is integrally and rigidly a part of foot 12. Foot 12 has a toe 40 and heel 42.

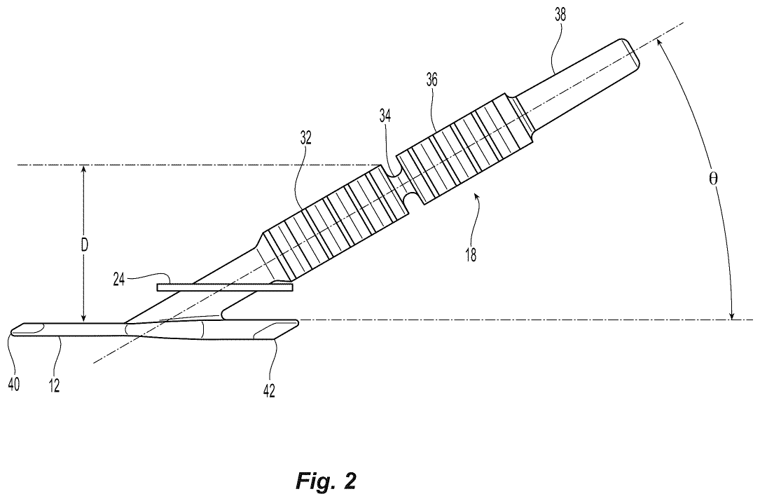

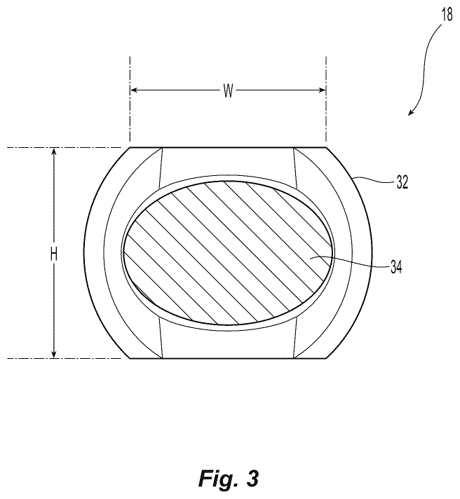

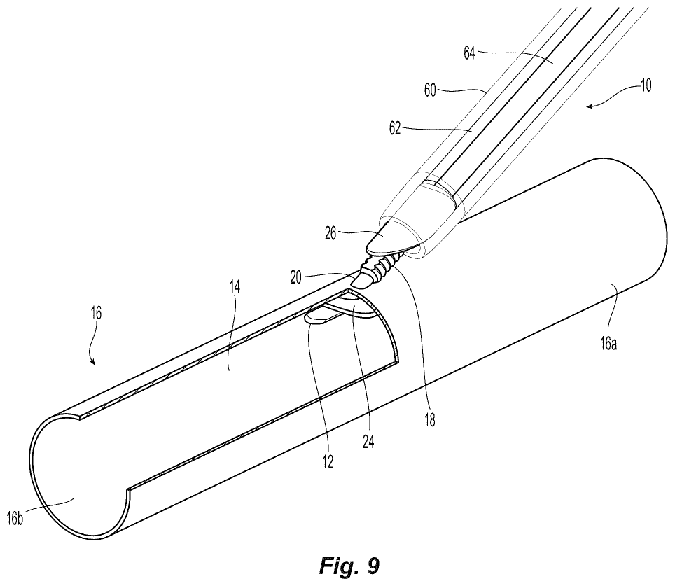

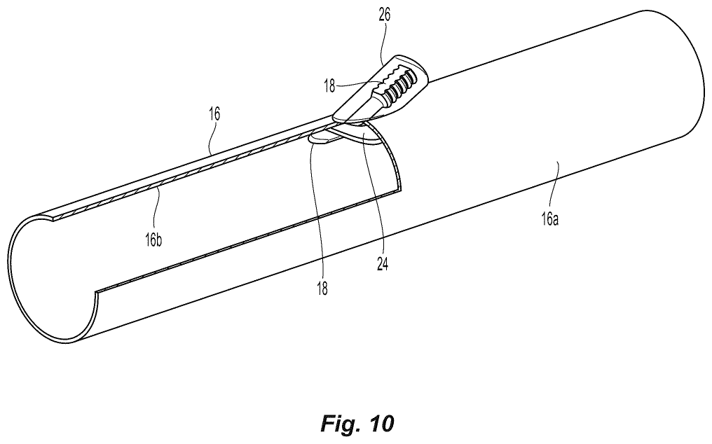

The shaft 18, particularly where the distal latches 32 and the proximal latches 36 are located, preferably has a width W that is greater than a height H. See FIG. 3. FIG. 3 shows the shaft 18 at the level of the break notch 34. The distal latches 32 and the proximal latches 36 are on the sides of the shaft 18. With the distal latches 32 being on the sides of the shaft 18 rather than on the top and bottom, the shaft 18 can make a smaller angle Ø relative to the foot 12. See FIG. 2. The smaller angle Ø allows for the vessel seal assembly 10 to be used where there is less tissue between the blood vessel and the skin of the patient. That is the distance D in FIG. 2. There needs to a certain number of distal latches 32 to securely retain the second sealing element 26 on the shaft 18 to close the opening in the blood vessel after deployment. The ankle 30 also provides a space for the wall of the blood vessel 16. See FIG. 9.

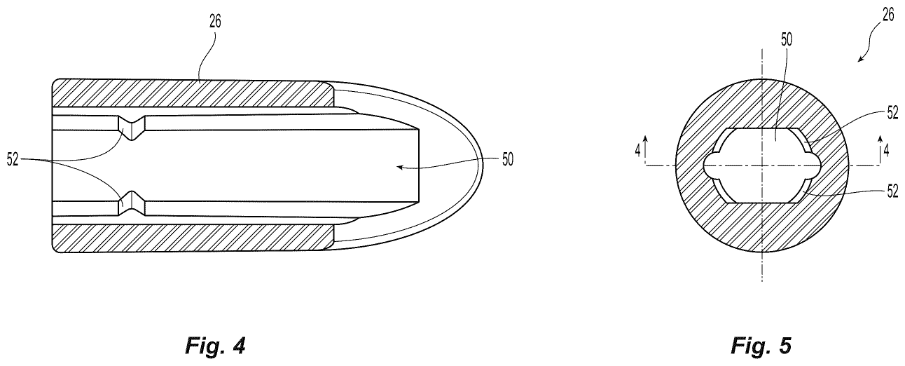

The second sealing element or proximal seal 26 is illustrated in FIGS. 4-6. The proximal seal 26 has a central opening 50 to receive the shaft 18. There are projections 52 that extend into the central opening 50 to engage the distal latches 32 and the proximal latches 36. The proximal seal 26 has a distal surface 54 that engages the outside of the blood vessel 16. The distal surface 54 has an angle Ø with the central opening 50, the angle Ø for the distal surface 54/shaft 18 is the same as the angle Ø for the shaft 18/foot 12. This configuration means that the first sealing member 12 and the distal surface 54 are parallel to one another and present the best chance to seal the opening 20 in the blood vessel 16.

The components of the vessel seal assembly 10, the foot 12, the shaft 18, the flexible member 24, and the proximal seal 26 are all made from a bio-absorbable material.

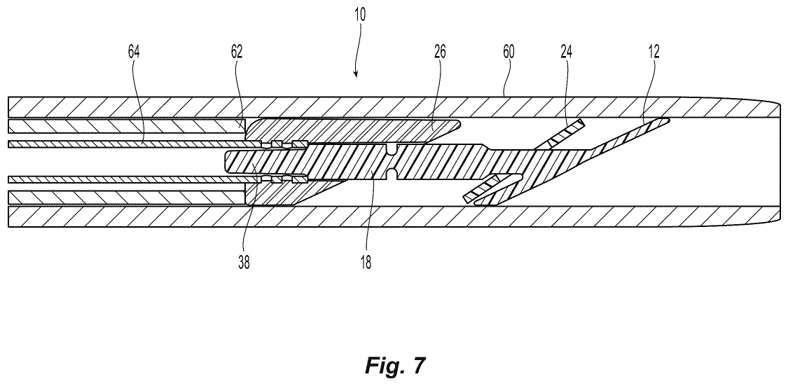

As illustrated in FIG. 7, the vessel seal assembly 10 fits within the sheath 60 without any deformation of any of the vessel seal assembly 10 or its components. A pusher tube 62 abuts the proximal seal 26, which is slidably attached to shaft 18. The distal end of a holding tube 64 is rigidly attached to holding taper 38 and the proximal end of the holding tube 64 is rigidly attached to the handle of the applier (not shown).

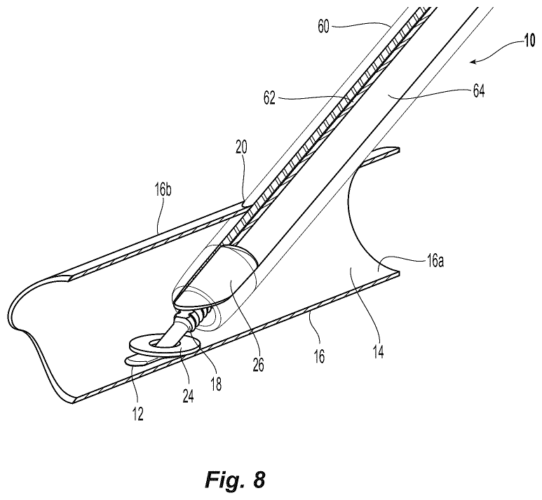

One embodiment of the present invention in the home state is illustrated in FIG. 8 with the vessel seal assembly 10 inserted into a shallow vessel 16 with an interior wall surface 16a and exterior wall surface 16b.

To deploy the vessel seal assembly 10, the sheath 60 and attached sealing device are first retracted proximally until flexible member 24 abuts interior wall surface 16a. See FIG. 9. A distal actuation force placed on pusher tube 62 causes the proximal seal 26 to move distally until the actuation force is countered by the seal sandwich force when the distal surface 54 of proximal seal 26 encounters exterior wall surface 16b. The flexible member 24 is drawn tightly against interior wall surface 16a as shown in FIG. 9. Increasing the actuation force further increases the sandwich force until it equals the break force of the shaft 18, thus detaching the foot 12, the flexible member 24, and the proximal seal 26 from the applier and leaving the sandwich force of the seal at the BN break force, which is independent of the user or the anatomy and equal to the BN break force.

The configuration of the vessel seal assembly 10, such that the facing areas of the proximal seal and the distal seal remain parallel from the home state throughout the proximal seal 26 travel to the seal state with no bending components allows for a more robust seal and eliminates the need for a FF element and results in instant hemostasis.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed:

1. A vessel seal assembly for sealing an opening in a wall of a blood vessel of an appendage such as the ulnar or radial artery, the blood vessel having an interior wall surface, exterior wall surface, and a lumen, the seal assembly comprising:
a first sealing element for placing inside the lumen of the blood vessel;
a shaft formed with the first sealing element as a single one-piece component, the shaft fixed in a predetermined configuration relative to the first sealing element, the shaft having a length sufficient to extend through the opening of the blood vessel and at least a portion of any tissue overlying the blood vessel;
a flexible member surrounding at least a portion of the shaft adjacent the first sealing element; and
a second sealing element, the second sealing element slidingly movable relative to the first sealing element along the shaft to engage the exterior wall surface and configured to position the flexible member against the interior wall surface of the blood vessel to seal the opening in the blood vessel.

2. The seal assembly for sealing an opening in the wall of a blood vessel according to claim 1, wherein the shaft and the first sealing element are joined at an angle between about 20 and 30 degrees.

3. The seal assembly for sealing an opening in the wall of a blood vessel according to claim 1, wherein the shaft and the first sealing element are joined at an angle of about 25 degrees.

4. The seal assembly for sealing an opening in the wall of a blood vessel according to claim 1, the second sealing element is configured to position the flexible member and the first sealing element against the interior wall surface of the blood vessel to seal the opening in the blood vessel.

5. The seal assembly for sealing an opening in the wall of a blood vessel according to claim 1, wherein the flexible member is connected to the first sealing element.

6. The seal assembly for sealing an opening in the wall of a blood vessel according to claim 1, wherein the flexible member is secured to a proximally facing surface of the first sealing element.

7. The seal assembly for sealing an opening in the wall of a blood vessel according to claim 1, wherein the flexible member is substantially circular and has an opening in a middle portion thereof to receive the shaft therethrough.

8. The seal assembly for sealing an opening in the wall of a blood vessel according to claim 1, wherein the flexible member has a shape selected from the shapes consisting of round and oval.

9. The seal assembly for sealing an opening in the wall of a blood vessel according to claim 1, wherein the shaft has a plurality of equally spaced projections along a length thereof.

10. The seal assembly for sealing an opening in the wall of a blood vessel according to claim 1, wherein the shaft has a reduced portion, the reduced portion having a cross section being smaller than a cross section of any other portion of the shaft.

11. The seal assembly for sealing an opening in the wall of a blood vessel according to claim 1, wherein the shaft has a plurality of equally spaced projections along a length thereof and the shaft has a reduced portion, the reduced portion having a cross section being smaller than a cross section of any other portion of the shaft.

12. The seal assembly for sealing an opening in the wall of a blood vessel according to claim 1, wherein the first sealing element, the shaft, the flexible member, and the proximal seal made from a bio-absorbable material.

13. The seal assembly for sealing an opening in the wall of a blood vessel according to claim 10, wherein the shaft breaks at the reduced portion as a result of a force exerted on the second sealing element, pushing the second sealing element against the blood vessel and the flexible member and the first sealing element.

14. The seal assembly for sealing an opening in the wall of a blood vessel according to claim 10, wherein a vertical distance from the reduced portion to a distal surface on the first sealing element is less than 5 mm.

15. The seal assembly for sealing an opening in the wall of a blood vessel according to claim 1, wherein the reduced portion is disposed between two of the plurality of equally spaced projections.

16. The seal assembly according to claim 9, wherein the second sealing element is latched into place when the reduced portion breaks, thus forming a sandwich force between the first sealing element and the second sealing element.

17. The seal assembly according to claim 16, where the sandwich force is dictated by strength of material and dimensions of the notch and the sandwich force between the first sealing element and the second sealing element is independent of a user or anatomy.
